(19) United States
(12) Patent Application Publication
(10) Pub. No.: US 2026/0157846 A1
(43) Pub. Date: Jun. 11, 2026

(54) HIGH DEFINITION AND EXTENDED DEPTH OF FIELD VIA SUBSURFACE MODIFICATION OF INTRAOCULAR LENS

(71) Applicant: Z Optics, Inc., Cookeville, TN (US)

(72) Inventor: Matthew Schuster, Cookeville, TN (US)

(21) Appl. No.: 18/702,625

(22) PCT Filed: Oct. 19, 2022
(86) PCT No.: PCT/US2022/047104

§ 371 Date: Apr. 18, 2024

Related U.S. Application Data
(60) Provisional application No. 63/257,405, filed on Oct. 19, 2021.

(51) Int. Cl. A61F 2/16

(52) U.S. Cl. CPC A61F 2/1616; A61F 2/1656

(57) ABSTRACT

An intraocular lens configured to provide an extended depth-of-field. The lends includes a virtual aperture that includes a first subsurface region having a first refractive index, and a plurality of modified, subsurface loci. The modified, subsurface loci have a second refractive index, which is different from the first refractive index. The loci can be configured to diffract light so as to widely disburse light across a retina.

### CROSS-REFERENCE TO RELATED APPLICATIONS

This application claims priority to U.S. Patent Application No. 63/257,405, filed Oct. 19, 2021, entitled “HIGH DEFINITION AND EXTENDED DEPTH OF FIELD VIA SUBSURFACE MODIFICATION OF INTRAOCULAR LENS”, the contents of which are hereby incorporated by reference herein in their entirety.

### BACKGROUND

The human eye often suffers from aberrations such as defocus and astigmatism that must be corrected to provide acceptable vision to maintain a high quality of life. Correction of these defocus and astigmatism aberrations can be accomplished using a lens. The lens can be located, for example, at a spectacle plane, at the corneal plane (a contact lens or corneal implant), or within the eye as a phakic (crystalline lens intact) or aphakic (crystalline lens removed) intraocular lens (IOL).

In addition to the basic aberrations of defocus and astigmatism, the eye often has higher-order aberrations such as spherical aberration and other aberrations. Chromatic aberrations, which are generally aberrations due to varying focus with wavelength across the visible spectrum, are also present in the eye. These higher-order aberrations and chromatic aberrations negatively affect the quality of a person's vision. The negative effects of the higher-order and chromatic aberrations increase as the pupil size increases. Vision with these aberrations removed is often referred to as high definition (HD) vision.

Presbyopia is the condition where the eye loses its ability to focus on objects at different distances. Aphakic eyes have presbyopia. A standard monofocal IOL implanted in an aphakic eye restores vision at a single focal distance. A variety of devices and procedures are used to provide improved vision over a range of distances, among them, using a monofocal IOL combined with bi-focal or progressive addition spectacles. A monovision IOL system is another option to restore near and distance vision-one eye is set at a different focal length than the fellow eye, thus providing binocular summation of the two focal points and providing blended visions. Monovision is currently the most common method of correcting presbyopia by using IOLs to correct the dominant eye for distance vision and the non-dominant eye for near vision in an attempt to achieve spectacle-free binocular vision from far to near.

Additionally, IOLs can be multifocal, for example, bifocal (having two focal regions-usually far and near) or trifocal (having three focal regions-far, intermediate, and near). Most multifocal IOLs are designed to have one or more focal regions distributed within an addition range. However, using elements with a set of discrete foci is not the only possible strategy of design: the use of elements with extended depth of field (EDOF), that is, elements producing a continuous focal segment spanning the required addition, can also be considered. These methods are not entirely acceptable as stray light from the various focal regions degrade a person's vision.

### SUMMARY

Disclosed are systems, devices, and methods that overcome limitations of IOLs at least by providing a phakic or aphakic IOL that simultaneously provides correction of defocus and astigmatism, decreases higher-order and chromatic aberrations, and provides an extended depth of field to improve vision quality. In addition, a central optic of the IOL provides a small “add” sector to increase the quality of vision corresponding to objects in a “near vision” region.

The disclosed IOL has an optical configuration that allows central focused light to reach the central focal area of the retina and spread defocused and aberrated light to a periphery of the retina. In one or more IOL regions where defocused and aberrated light is widely spread across the retina, high power refractive and/or total internal reflection is employed. The result is an optical configuration that increases depth of focus and decreases monochromatic and chromatic aberrations providing high definition vision over a wide range of object distances from far to near vision.

In one aspect, there is disclosed an intraocular lens configured to provide an extended depth-of-field, said intraocular lens comprising: an optical zone comprising at least one anterior optical surface and at least one posterior optical surface; a first periphery region peripherally positioned relative to the optical zone, the first periphery region comprising a virtual aperture, the virtual aperture comprising an anterior virtual aperture surface and a posterior virtual aperture surface, wherein the virtual aperture comprises a first subsurface region having a first refractive index, and wherein the virtual aperture further comprises a plurality of modified, subsurface loci, wherein the modified, subsurface loci have a second refractive index, which is different from the first refractive index and caused by nonlinear absorption of photons resulting from exposure to focused laser light; and a second periphery region peripherally positioned relative to the first periphery region, the second periphery region comprising a haptic for positioning the intraocular lens within an eye, wherein the haptic comprises an outermost region of the intraocular lens; wherein a first plurality of light rays incident on the anterior optical surface pass through the optical zone to form an image on a retina when the intraocular lens is implanted in an eye; and wherein a second plurality of light rays incident on the anterior virtual aperture surface are dispersed widely downstream from the intraocular lens towards and across the retina, such that the image comprises the extended depth-of-field and further wherein said virtual aperture reduces monochromatic and chromatic aberrations in the image.

The details of one or more variations of the subject matter described herein are set forth in the accompanying drawings and the description below. Other features and advantages of the subject matter described herein will be apparent from the description and drawings, and from the claims.

### BRIEF DESCRIPTION OF THE DRAWINGS

FIGS. 1A and 1B illustrates a basic method of reducing monochromatic aberrations and increasing or extending depth of field using pupil size for a near-sighted eye.

FIGS. 2A and 2B illustrate a basic method of reducing monochromatic aberrations and increasing depth of field using pupil size for a far-sighted eye.

FIGS. 3A and 3B illustrate a basic method of reducing monochromatic aberrations and increasing depth of field using pupil size for an emmetropic eye.

FIGS. 4A and 4B illustrate the basic method of reducing chromatic aberrations using pupil size.

FIGS. 5A and 5B illustrate the basic concept of the virtual aperture to limit the effective pupil size.

FIGS. 6A, 6B, and 6C illustrates an overall structure of an example IOL.

FIG. 6D shows another embodiment of an example IOL.

FIG. 7 illustrates an example IOL with hexagonal sampled micro-lenses in a virtual aperture zone.

FIG. 8 illustrates an example hexagon geometry of a micro-lens.

FIG. 9 illustrates a center portion of a two-dimensional array of hexagons of micro-lenses.

FIGS. 10A and 10B illustrate two neighboring hexagons, the neighboring corresponding micro-lens spheres, and a smooth surface profile that supports a minimum curvature.

FIG. 11 Illustrates an example partition of an optical zone of an IOL to provide both near and distance vision partitions.

FIGS. 12-14 relate to systems for modifying a subsurface region of an IOL.

### DETAILED DESCRIPTION

Before the present subject matter is further described, it is to be understood that this subject matter described herein is not limited to particular embodiments described, as such may of course vary. It is also to be understood that the terminology used herein is for the purpose of describing particular embodiments only, and is not intended to be limiting. Unless defined otherwise, all technical terms used herein have the same meaning as commonly understood by one skilled in the art to which this subject matter belongs.

Disclosed are systems, devices, and methods that overcome limitations of IOLs at least by providing a phakic or aphakic IOL that provides correction of defocus and astigmatism, decreases higher-order monochromatic and chromatic aberrations, and provides an extended depth of field to improve vision quality. The disclosed IOL is sometimes referred to herein as the Z+ optic or Z+IOL. PCT application PCT/US20/37014 describe related systems and methods and is incorporated herein by reference in their entirety.

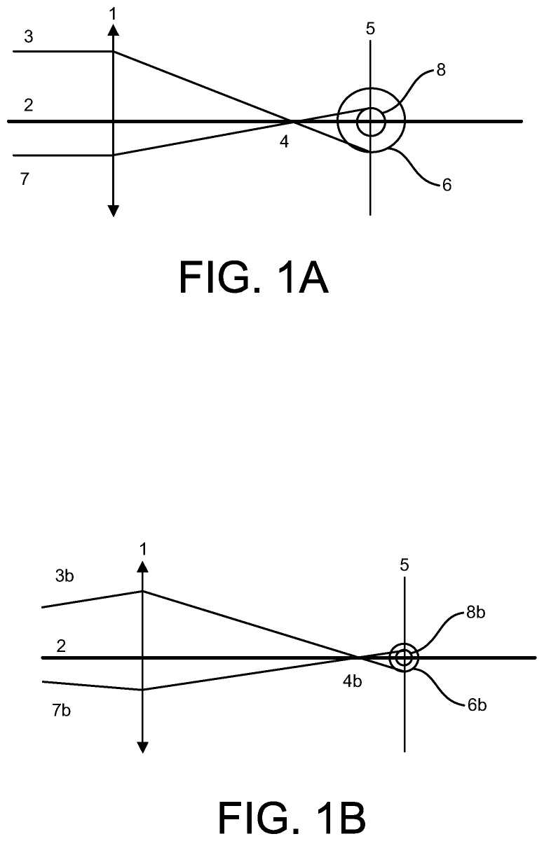

A description of the basic principle used to reduce monochromatic and chromatic aberrations and provide an increased depth of field is now provided. FIG. 1A schematically illustrates a single converging lens 1 centered on an optical axis 2. An incident ray 3 from a distant object is parallel to the optical axis and intersects the focal point 4 (with a suffix b, c, d, e, or f based on the corresponding figure) of the lens. If the lens power is properly selected, the focal point coincides with the observation plane 5, otherwise there is a mismatch between the lens power and the location of the observation plane such that the focus is in front of or behind the observation plane.

In FIG. 1A, the focal point is in front of the observation plane. If all incident rays are traced with the same ray height as incident ray 3, a blur circle 6 is located on the observation plane 5. The observation plane is oriented orthogonal to the optical axis and so is shown as a vertical line in the figure. The blur circles 6 and 8 are shown in the plane of the figure for visualization convenience, however, the blur circles are actually contained in the observation plane. Other parallel incident rays with ray height less than incident ray 3 fall inside this blur circle 6. One such ray is parallel incident ray 7 which is closer to the optical axis than incident ray 3. Incident ray 7 also intersects the focal point 4 and then the observation plane 5. Tracing all incident rays with ray height equal to incident ray 7 traces out blur circle 8 which has a diameter smaller than that of blur circle 6.

FIG. 1B illustrates the same optical system in FIG. 1A, but now the incident rays are for an object closer to the optical system as indicated by the slopes on incident rays 3b and 7b. The effect is that the focus point 4 (with a suffice a, b, c, d, or f based on the corresponding figure) for the closer object is now closer to the observation plane and both of the blur circles 6b and 8b are smaller than their counter parts in FIG. 1A, but the principle is the same: rays which intersect the lens 1 closer to the optical axis have smaller blur on the observation plane. To relate this simple optical construction of FIG. 1 to the human eye, the converging lens 1 represents the principal plane of the eye's optics including the cornea and the crystalline lens or an intraocular lens. Observation plane 5 represents the retina. As drawn the focal point 4 is in front of the observation plane (retina), so this figure is for a myopic or near-sighted eye. The size of the blur circles 6 and 8 (or 6b and 8b) represents the amount of defocus on the retina, where a smaller blur circle diameter provides clearer vision than a larger blur circle diameter.

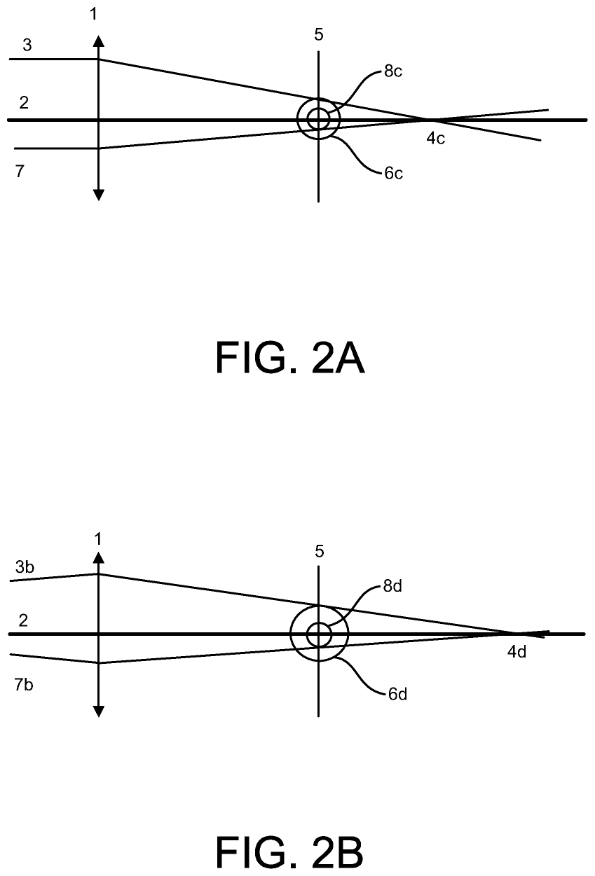

Note that the same relationship regarding incident ray height and blur circle size also holds for hyperopic or far-sighted eyes. This is schematically illustrated in FIGS. 2A and 2B, which show rays corresponding to a far-sighted eye. In FIG. 2A for rays 3 and 7 from a distant object and in FIG. 2B for rays 3b and 7b, smaller ray height leads to a smaller blur circle on the retina (observation plane).

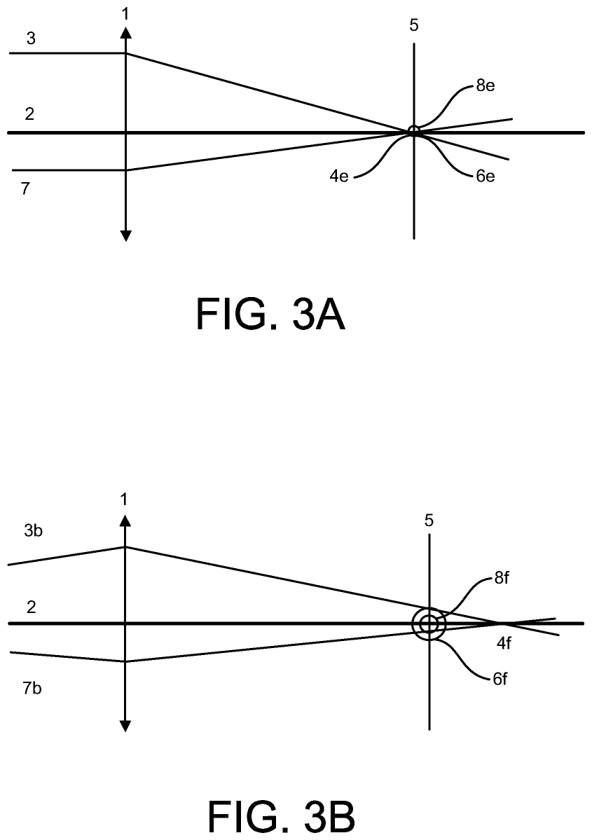

Similarly, FIGS. 3A and 3B (collectively referred to as FIG. 3) show that the same parallel ray height to blur circle diameter property holds for an emmetropic eye. For a distant object, the focal point 4e is now at the retina (since the eye is emmetropic) and the blur circles 6e and 8e have zero radius. For a closer object, the focal point 4f is behind the retina and blur circle 8f corresponding to ray 7b which is closer to the optic axis has a smaller diameter than blur circle 6f corresponding to ray 3b which is further from the optic axis.

In general, an eye has aberrations, which means that as an incident ray location changes, the focal point in the eye also changes. But regardless of where the focal points are located (in front of-, on-, or behind the retina), as incident ray heights are reduced so are the blur circle diameters on the retina. Stated another way, for a given amount of defocus (dioptric error) in the eye, vision is improved as the height of incident rays is reduced. This principle is used when someone squints causing the eyelids to block the incident rays further from the optic axis of the eye in an attempt to see an out-of-focus distant or near object more clearly.

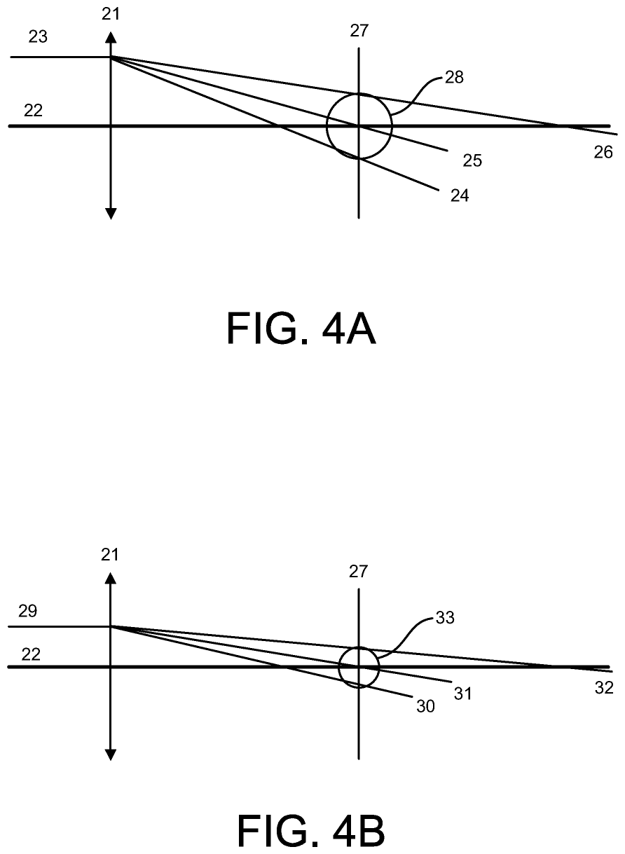

The ray tracing illustrated in FIGS. 1A-3B is for a single wavelength of incident light. For polychromatic light, multiple wavelengths are present. This is commonly illustrated by three rays of different wavelengths as shown in FIGS. 4A and 4B (collectively referred to as FIG. 4). It is well known that for the components of the eye and typical optical materials, as the wavelength of light increases, the refractive index decreases.

In FIG. 4A, a converging lens 21 has optical axis 22. An incident chromatic ray 23 consists of three wavelengths for blue (450 nm), green (550 nm), and red (650 nm) light which approximately span the range of visible light. Due to different indices of refraction for the three wavelengths, the blue light ray 24 is refracted more than the green light ray 25, and the green light ray is refracted more than the red light ray 26. If the green light ray is in focus, then it crosses the observation plane 27 at the optical axis. The chromatic spread of these three rays lead to a chromatic blur 28 on the observation plane.

In FIG. 4B, the incident chromatic ray 29 has a lower ray height than the chromatic ray 23 in 4A. This leads to smaller chromatic blur 33 at the observation plane. Thus, just as for the monochromatic blur of FIGS. 1A-3B, chromatic blur is decreased as the chromatic ray height is decreased. The situation in FIG. 4 can be related to the eye by considering converging lens 21 to be the principal plane of the eye and observation plane 27 to be the retina. The human eye normally has a large amount of chromatic aberration (about 1.0 to 1.2 diopters over the central visual range) so this reduction in chromatic aberration can be significant leading to a noticeable improvement in the eye's visual quality, especially as measured by its contrast sensitivity.

Taken together, FIGS. 1A-4B illustrate that decreasing ray height decreases both monochromatic and chromatic aberrations at the retina, thus increasing the quality of vision. This can be accomplished by either blocking rays with larger distance from the optical axis by decreasing the pupil diameter or by spreading light from these rays evenly and/or widely across the retina so that more aberrant rays contribute much less light to the central retinal blur circle. Another feature of this effect is that the depth of field is increased as the ray height is decreased as illustrated in FIGS. 1B, 2B, and 3B.

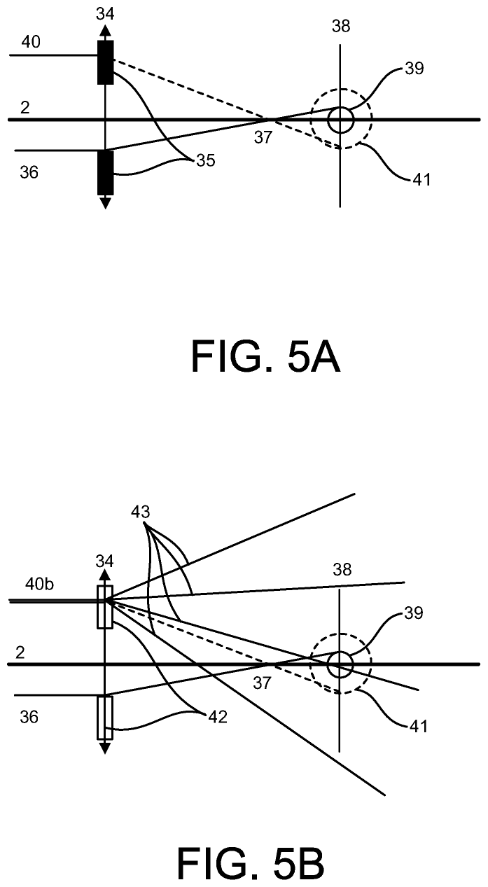

FIG. 5A shows a converging lens 34 with optical axis 2 and aperture 35. Incident parallel ray 36 just clears the aperture and thus passes through the lens focal point 37 and intersects the observation plane 38. All parallel rays with the same height as ray 36 trace a small blur circle 39 on the observation plane. Incident parallel ray 40 is blocked by the aperture, and thus it cannot continue to the observation plane to cause a larger blur circle 41. In this way, an aperture which reduces the incident ray height reduces the blur diameter on the observation plane.

FIG. 5B illustrates a “virtual aperture”. That is, it is not really an aperture that blocks rays, but the optical effect is nearly the same on central vision. In this figure, bundle of rays 40b incident on the virtual aperture propagate through the virtual aperture 42 and through refraction, diffraction, scattering, reflection, and/or diffusion yield rays 43 which are widely spread out so there is very little contribution to stray light (blurring light) at any one spot on the observation plane. This is a principal mechanism of operation of the disclosed IOL.

One type of a light altering mechanism in the virtual aperture is a plurality of holes of predetermined size positioned on a region or an entirety of the virtual aperture. The holes are individually or collectively configured to permit the passage of light therethrough such a to create diffractive effects and/or to achieve or vary a desired level of opacity. The holes can have a spatial arrangement/pattern, diameter, depth, and/or density of hole patterns that enables a specific light scattering pattern relative to the retina to achieve desired diffusive effects, such as, for example, homogeneous distribution of light and/or scattering of light. In an embodiment, the holes have a density across the virtual aperture so as to achieve light transparency in the range of 10 percent to 100 percent light transparency in the virtual aperture with the holes being arranged to achieve this.

### Exemplary Optical Layout of the IOL

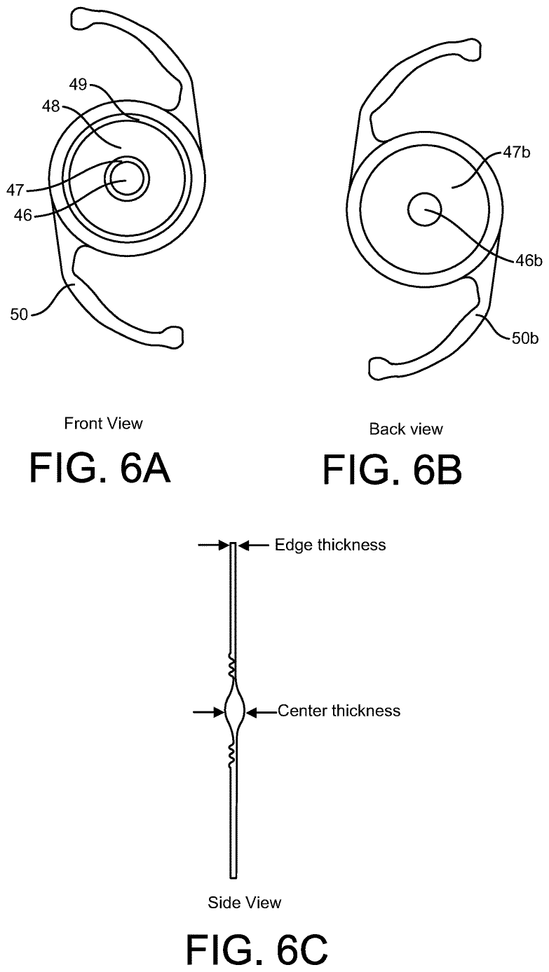

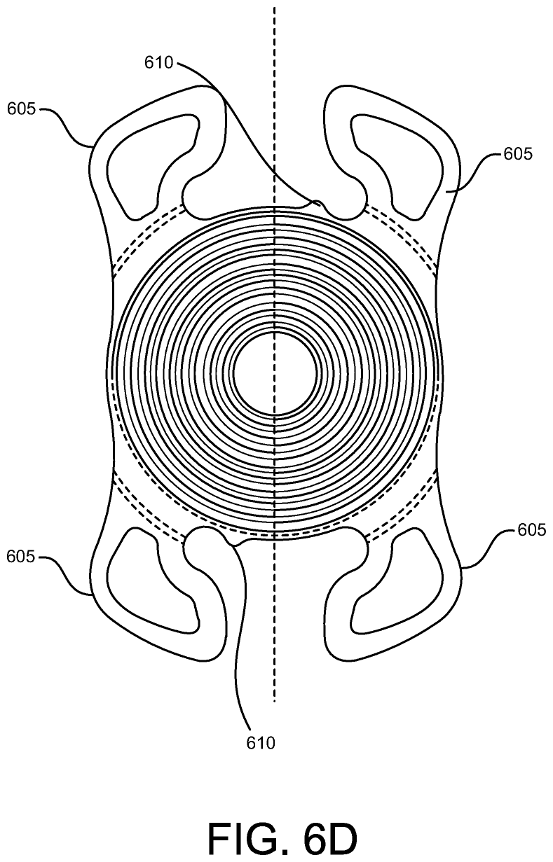

FIGS. 6A-6C illustrate a layout of an example IOL that employs optical principles to achieve the benefits of decreased monochromatic and chromatic aberrations and increased depth of field. FIG. 6A shows a front view of the IOL wherein the front view may be an anterior view. FIG. 6B shows a back view of the IOL wherein the back view may be a posterior view. FIG. 6C shows a side view of the IOL. The IOL includes a central optical zone 46 (with back side 46b) that provides correction of defocus, astigmatism, and any other correction required of the lens such as spherical aberration. Generally, for an IOL using a virtual aperture, the central optical zone diameter is smaller than that of a traditional IOL. This leads to a smaller central thickness which in turn makes the IOL easier to implant and allows a smaller corneal incision during surgery, such as an incision on the order of 2.2 mm.

The IOL includes a virtual aperture 48 that is positioned further peripherally outward relative to the center location of the central optical zone 46. Moving peripherally outward from the virtual aperture 48, at least one IOL haptic 50 (with back side 50b) is located on the IOL. The haptic 50 can be formed of one or more arms that extend peripherally outward to define a peripheral most edge of the IOL. In a non-limiting example, the optical zone has a diameter of 1.5 mm. A 1.5 mm diameter optical zone has been shown to be at or near the minimum threshold required to allow enough light into the eye under mesonic conditions. In other non-limiting examples, the diameter of the optical zone is 1.5 mm to 2.8 mm or 3 mm in size. In another embodiment, the optical is 1.5 to 3.3 mm in size wherein the size corresponds to a diameter. In other non-limiting examples, the diameter of the optical zone is 1.65 mm or 3.3 mm. The haptic 50 may define an outermost peripheral region of the IOL. A first plurality of light rays incident on an anterior optical surface of the optical zone can pass through the optical zone to form an image on a retina when the IOL is positioned in an eye, while a second plurality of light rays incident on an anterior virtual aperture surface are dispersed widely downstream from the IOL towards and across the retina, such that the image comprises an extended depth-of-field and further wherein the virtual aperture reduces monochromatic and chromatic aberrations in the image. The optical zone can comprise at least one of bifocal optics, trifocal optics and multifocal optics.

The virtual aperture is connected to the optical zone 46 by a first transition region 47, which is located at a peripheral edge of the optical zone 46 such that the virtual aperture is a first periphery region that surrounds or partially surrounds the optical zone. The haptic can comprise a second periphery region for positioning the intraocular lens within an eye. The first transition region is located peripherally outward of the optical zone 46. A second transition region 49 connects the haptic 50 to the virtual aperture 48. The first transition region 47 and the second transition region 49 are configured to ensure zero- and first-order continuity of an outer surface of the IOL on either side of the respective transition region. A common way to implement these transition regions is a polynomial function such as a cubic Bezier function. Transition methods such as these are known to those skilled in the art. On the back side of the IOL is a central optic zone 46b, a haptic 50b, and a transition 47b between them. FIGS. 6A-6C are not necessarily to scale, and the haptic shape is for illustration purposes only. Other haptic shapes and sizes known to those skilled in the art would be suitable as well. The first and second transition regions are not necessarily present per se in the IOL.

The IOL has an anterior surface and a posterior surface and the components of the IOL including the optical zone 46, the first transition region 47, the second transition region 49, the virtual aperture 48, the haptic 50 can each have a respective anterior surface and posterior surface. The optical zone 46 has an anterior optical surface that can include at least one multifocal zone and/or a toric region. At least a portion or region of the anterior surface and/or the posterior surface, such as in the region of the virtual aperture or other portion of the IOL, can have a surface contour or shape that achieves a desired or predetermined effect for light passing therethrough. In nonlimiting examples, the surface contour of the anterior surface and/or the posterior surface includes a region with a ripple-type contour such as a wave shape or an undulating shape that forms a series of raised and lowered surfaces. The surface contours can achieve various effects with respect to light passing through the IOL. For example, the surface contour can achieve a wide or wider spread of stray light depending upon the type of surface contour used. The surface contour can be used to achieve a spread of stray light which is guided away from a focal point of the retina.

FIG. 6D shows a front view of another embodiment of an IOL, which includes a central optical zone, a plurality of peripheral haptics 605, and at least one zone having a surface contour such as a ripple or wave as described further below. In an example, the optical zone has a diameter of 1.5 mm and serves as a lens which brings distant objects into sharp focus on the central retina.

The IOL includes one or more orientation structures 610 such as one or more protrusions or nubs. In the illustrated embodiment, the orientation structures 610 are positioned on a peripheral edge of a portion of the IOL with at least one orientation structure 610 on the first side of a vertical meridian of the IOL and a second orientation structure 610 on a second side of the vertical meridian. Meridian. The vertical meridian is shown as a dashed line in FIG. 6D. The orientation structures 610 are configured to allow a clinician, such as a surgeon, to easily detect that the IOL has a correct side facing the front of the eye. Note that if the IOL were oriented with the back side facing the front of the eye, the orientation structures 610 would be counter-clockwise with respect to the vertical of the lens.

A discussed, the haptic(s) 605 provide a mechanical interface with the eye and holds the various zones of the IOL at its proper position relative to the eye.

### Example Optic Zone Details—Hexagonal Micro-Lens Virtual Aperture

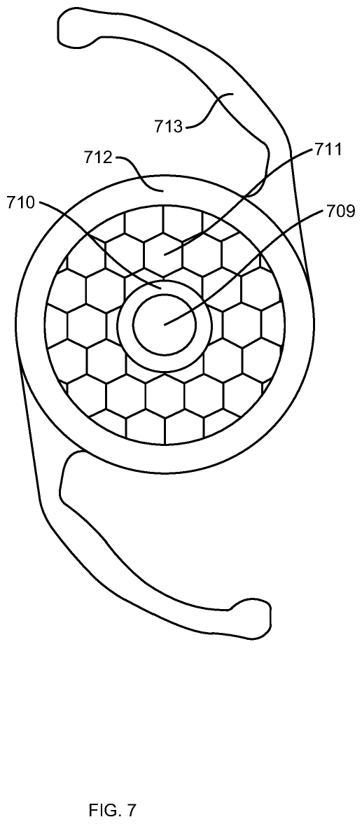

FIG. 7 illustrates a front view of an IOL that includes a virtual aperture having one or more hexagonal structures. The IOL has central optical zone 709, a first transition zone 710, a hexagonal micro-lens virtual aperture 711, a second transition zone 712, and a haptic 713. The first transition zone 710 connects the central optical zone 709 to the hexagonal micro-lens virtual aperture 711 while the second transition zone 712 connects the hexagonal micro-lens virtual aperture 711 to the haptics 713.

The virtual aperture employs a two-dimensional hexagonal sampled array of micro-lenses which mimics the photo sensor sampling of the retina. This arrangement is a beneficial layout for widely spreading light across the retina when the IOL is implanted in an eye.

The hexagonal micro-lens virtual aperture 711 include a plurality of hexagonal shaped microstructures positioned on a front side and/or a backside of the IOL. The hexagonal shape is with respect to an outer boundary of each hexagonal micro-structure has an outer boundary defined by a hexagon microstructure when viewed from a front or rear of the IOL. That is, a hexagonal micro-structure can have an outer boundary defined by a hexagon. A small lens is placed inside the bounds of each of the hexagonal micro-structures. The lens can be a structure that is positioned on or in the micro-structure. The lens may also be monolithically formed as part of the microstructure during manufacture. To help prevent unwanted patterning of light on the retina, the centers of micro-lenses inside each hexagon are randomly moved or positioned on the IOL, and the radii of the micro-lenses are also adjusted. To facilitate manufacturing of the hexagonal micro-lens virtual aperture, between the hexagon boundaries of the micro-lenses, a blending region or fillet is placed with a radius of curvature greater than the radius of a lathe cutter that forms the micro-lens. This radius is on the order of 0.05 mm in a non-limiting example.

The hexagon can have a variety of dimensions. In an embodiment, the hexagon of a micro-structure is more tall than wide. In another embodiment, the hexagon of a micro-structure is more wide than tall. In another embodiment, the outer boundary of a micro-structure is an arbitrarily-shaped polygon.

With reference still to FIG. 7, the first transition zone 710 is configured to provide a smooth structural blend between the edge of the optical zone 709 and the central hexagonal micro-lens region 711. The second transition zone 712 is responsible for providing a smooth structural blend between the peripheral hexagonal micro-lens region 711 and the haptic 713. These transition regions can be effectively accomplished using Bezier curves or portions of Bezier surfaces to define a surface of the respective zone. Other transition functions can be suitable as well and are known to those skilled in the art. It should be appreciated that any of the embodiments of the IOLs described herein can be configured to not include any transition zones.

The micro-lenses are implemented as one or more outer surfaces defined at least partially by a sphere, conicoid, or other similar outer surface that can achieve high optical power to widely spread incoming light rays across the retina. For example, the micro-lenses are implemented as one or more outer surfaces defined at least partially by a prismatic or pyramid shape. As an example, in the following discussion there are illustrated embodiments with spherical micro-lenses.

### Nominal Hexagonal Sampling

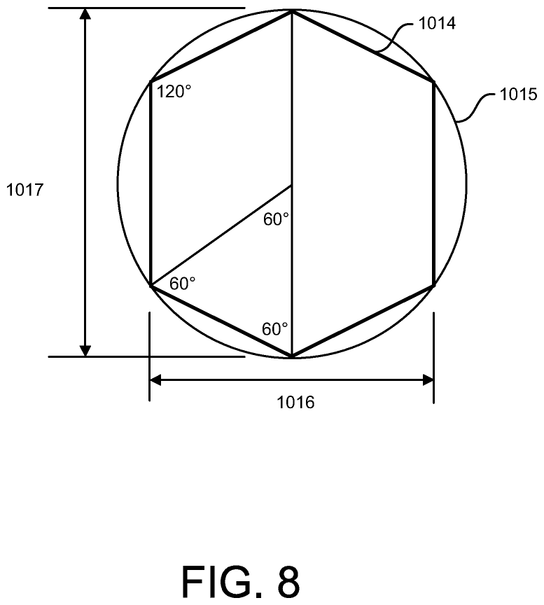

One example hexagon is illustrated in FIG. 8, which shows an example micro-structure of the virtual aperture with the micro-structure being defined by a hexagon 1014. In FIG. 8, the hexagon 1014 is shown inside a bounding circle 1015 that defines a shape or size of the hexagon. The hexagon has width 1016 and a height 1017. As illustrated, the height of the hexagon is equal to the diameter of the bounding circle 1015. In terms of the radius of the bounding circle, the width of the hexagon is found using Pythagoras's rule to be as given by equation (1) and the height is given by equation (2):

$$\begin{matrix}width = \sqrt{3} \times r & ( 1 ) \\ height = 2 \times r where r = radius of bounding circle & ( 2 )\end{matrix}$$

Also, (a) each interior angle of the hexagon is 120 degrees, (b) each side and the center point form an equilateral triangle with interior angle of 60 degrees, and (c) the hexagon side length is equal to the radius of the bounding circle.

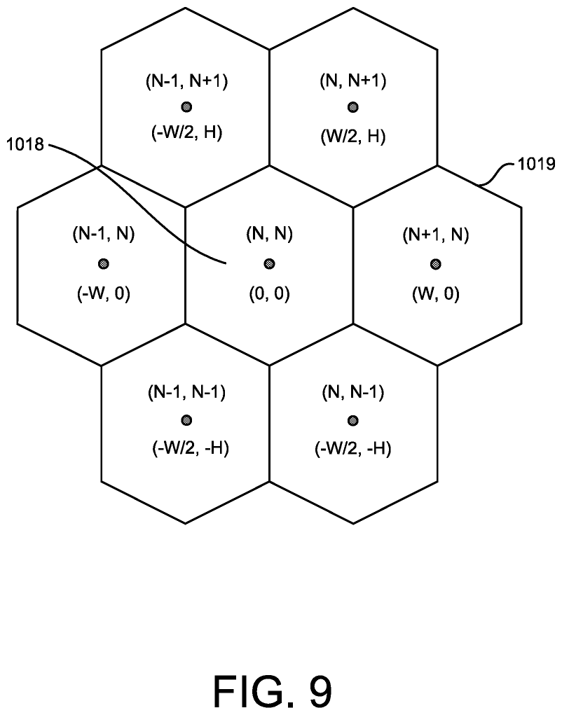

The center portion of a two-dimensional array of hexagons is illustrated in FIG. 9. The dimensions of the two-dimensional array of hexagons is defined by equation (3).

$$M \times M = ( 2 N + 1 ) \times ( 2 N + 1 ) \qquad ( 3 )$$

In this equation, N is a positive, even integer, for example, 50. The (x, y) location of the center of each hexagon is given by equations (4a) and (4b).

$$\begin{matrix}x = \{ \begin{matrix}( i - N ) \times width & for even i \\ ( i - N + \frac{1}{2} ) \times width & for odd i\end{matrix} & ( 4 a ) \\ y = ( j - N ) \times height & ( 4 b )\end{matrix}$$

The index of the two-dimensional hexagonal array elements as well as the (x, y) coordinates of the hexagon centers are illustrated in the pairs of values above and below each hexagon center in FIG. 9.

### Smooth Profiles Across the Micro-Lenses

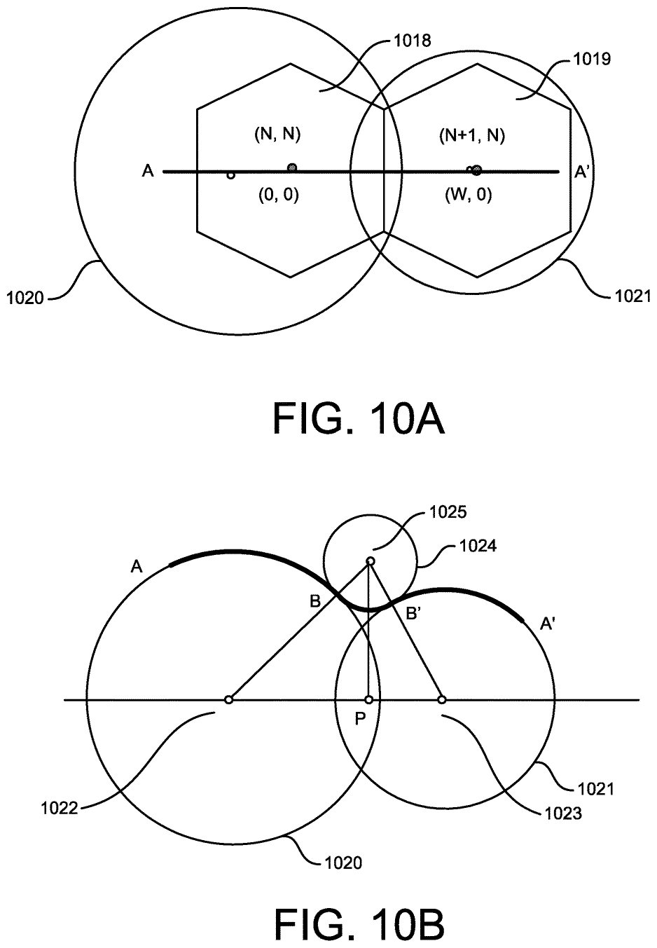

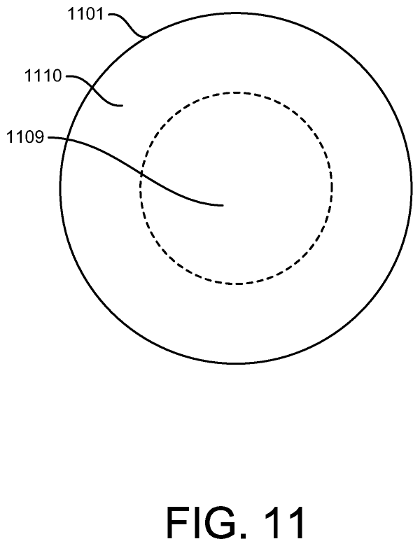

FIG. 10A illustrates two, example neighboring or adjacent hexagons at the center of the two-dimensional array. The center of this array can be in coincidence with the optical axis of the IOL. The hexagon 1018 has its center at the center of the optical axis of the IOL. A micro-lens spherical surface 1020 has its center located at a random (x, y) distance from the center of hexagon 1018. A hexagon 1019 is a direct neighbor of hexagon 1018 and a micro-lens spherical surface 1021 has its center located at a random (x, y) distance from the center of hexagon 1019. The radius of micro-lens spherical surface 1020 is larger than the radius of micro-lens spherical surface 1021. In FIG. 10A, the coordinates can be referred to as (x, y) with z coming out of the page, x directed to the right and y directed up. Thus, this represents a view looking down onto the surface of the lens and each micro-lens spherical surface is convex, thus producing a local positive high-power surface. FIG. 10A also shows is profile AA′ which extends through the centers of micro-lens spherical surfaces 1020 and 1021.

FIG. 10B illustrates the side view of the geometry shown in FIG. 10A. The spheres 1020 and 1021 are shown corresponding to the same spheres in FIG. 10A. The centers of the spheres are indicated as points 1022 and 1023, corresponding to spheres 1020 and 1021, respectively. The coordinates in this figure can be referred to as (x, z) with y into the page, x directed to the right and z directed up. Here it can be seen that the profile AA′ is a curve on the surface of the micro-lens array due to spheres 1020 and 1021 as well as a spherical fillet 1024. The spheres shown in FIG. 10B are convex and the fillet sphere is concave. In another example, the micro-lens spheres are concave, and the fillet sphere is convex. This latter orientation of the spheres provides the benefit that a smaller fillet sphere radius can be used that is not limited by the radius of the cutting tool.

Pursuant to a manufacturing process of an IOL, the radius of a spherical fillet is selected to be larger than the radius of a lathe cutting tool so that the surface can be generated with the given cutting tool. To find the surface points of the smooth profile AA′, the center 1025 of the spherical fillet 1024 is defined as a known radius. For simplicity the centers of the micro-lenses are constrained to have z value on a plane perpendicular to the optical axis. The point P shown in FIG. 10B has the same (x, y) coordinates as the center 1025 of fillet sphere 1024 and is located on the line connecting the micro-lens centers 1022 and 1023. The coordinates of the point P are given in equation (5a).

$$\begin{matrix}P = [ \begin{matrix}{P}_{x} \\ {P}_{y} \\ {P}_{z}\end{matrix} ] = [ \begin{matrix}{x}_{1} \\ {y}_{1} \\ {z}_{1}\end{matrix} ] + t [ \begin{matrix}a \\ b \\ c\end{matrix} ] where , & ( 5 a ) \\ a = {x}_{2} - {x}_{1} b = {y}_{2} - {y}_{1} c = {z}_{2} - {z}_{1} & ( 5 b ) \\ t = \frac{d - a {x}_{1} - b {y}_{1} - c {z}_{1}}{{a}^{2} + {b}^{2} + {c}^{2}} & ( 5 c ) \\ d = \frac{{R}_{1}^{2} - {R}_{2}^{2} - ( {x}_{1}^{2} - {x}_{2}^{2} ) - ( {y}_{1}^{2} - {y}_{2}^{2} ) - ( {z}_{1}^{2} - {z}_{2}^{2} )}{2} & ( 5 d )\end{matrix}$$

In these equations,

$${R}_{1}^{2} = the radius of the first sphere ( item 1020 ) plus the radius of the fillet sphere$$

$${R}_{2}^{2} = the radius of the second sphere ( item 1021 ) plus the radius of the fillet sphere$$

x₁, y₁, z₁=the center of the first sphere (item 1022)
x₂, y₂, z₂=the center of the first sphere (item 1023)

The set of center points for the center of the fillet sphere for the entire micro-lens spheres can then be found from equation (6a).

$$\begin{matrix}[ \begin{matrix}x ( θ ) \\ y ( θ ) \\ z ( θ )\end{matrix} ] = [ \begin{matrix}{P}_{x} \\ {P}_{y} \\ {P}_{x}\end{matrix} ] + r [ \begin{matrix}{u}_{x} cos ( θ ) \\ {u}_{y} cos ( θ ) \\ {u}_{z} sin ( θ )\end{matrix} ] where , & ( 6 a ) \\ w = \frac{[ \begin{matrix}a \\ b \\ c\end{matrix} ]}{\vert [ \begin{matrix}a \\ b \\ c\end{matrix} ] \vert} & ( 6 b ) \\ v = [ \begin{matrix}0 \\ 0 \\ 1\end{matrix} ] & \\ u = v \times w & \\ r = \sqrt{{R}_{1}^{2} - ( {a}^{2} + {b}^{2} + {c}^{2} ) {t}^{2}} & \end{matrix}$$

The angle θ is in the plane containing P and perpendicular to the line intersecting the two micro lens sphere centers. Using this geometry, there can be traced out the surface points along the curve segments AB, BB′, and B′A′. Together, these points form a continuous blending between each of the micro-lenses in the virtual aperture such that they can be cut on a lathe using a tool of radius less than the fillet sphere radius.

To use the concepts described above to define a surface of the IOL, the following is done. First, the central optic of the IOL is specified such as described in PCT Patent Application Serial No. PCT/US20/37014 and U.S. patent application Ser. No. 16/380,622, which are incorporated by reference in their entirety. The diameter of the optic zone can be around 1.5 mm and preferably between (1.4 and 1.6 mm) in non-limiting examples. Optical powers for this optic zone vary from −10 to 40 D in steps or 0.25 or 0.5 D. Cylinder powers for toric IOLs vary from 0.5 to 6.0 D in steps of 0.25 to 0.5 D.

The micro-lens array virtual aperture is then generated using the concepts above where the radius of the circles bounding the hexagons is about 0.125 mm. The centers of the individual micro-lens spheres are randomly varied about 0.05 mm in x and y. The radii of the micro-lens spheres are randomly varied 0.05 mm from a mean radius of about 0.2 mm. The width of the virtual aperture region is about 2.0 mm.

The micro-lens array fillet sphere radius is set to be about 25% larger than the lathe tool radius. This can be around 0.05 mm.

The width of the front surface transition regions is each set to around 0.15 mm. The width of the back-surface transition region is set to around 2.3 mm.

The configuration of the haptic is configured according to routine procedures for those skilled in the art.

Once the front and back surfaces have been specified, individual profile samples are taken from the center of the IOL to the periphery to specify the points for the lathe cutting file.

### Multi-Region Optical Zone

FIG. 11 schematically illustrates a multi-region, such as two-region, optical zone 1101 that can be included in any IOL described herein. The regions are indicated 1109 and 1110. These represent two distinct regions in the optical zone for two distinct powers. For example, a first discrete region is a central region 1109 is normally for providing distance vision. A second discrete region is a peripheral region 1110 is normally for providing near vision. The “add” of the near vision region is around 3.0D and in the range of 2.0 to 3.5D.

Due to the special nature of IOL's optical mechanism of action, providing a bifocal optical zone is not as problematic as normal size optical zones of 5.0 mm and larger. This is because the extra aberrations caused by incident rays which are outside the central optical zone diameter of, typically, 1.5 mm, are widely distributed across the retina so as not to negatively affect the central vision of the eye.

### Distribution of Optic Zone Regions

In an example configuration, the distance power region of the central optic takes up 75% of the optic zone area and the near power region of the central optic takes up 25% of the optic zone area. Since the diameter of the central optic zone is typically 1.5 mm, the central region 1109 of the optical zone has diameter 1.3 mm and the remainder of the optic zone provides 25% for the near vision region 1110.

For some eyes it can be preferred to have the distribution of distance region area and near region area portioned to 50% each or 25% for distance and 75% for near vision. Providing one eye with a majority of the optic zone area for distance vision, such as 75 to 100%, and the other eye with more area optical zone area for near vision may would be used for extended depth of focus/monovision patients. In this case, both eyes have extended depth of focus, but one eye (usually the dominant eye) has slightly better performance for distance vision and the other eye has slightly better visual performance for near vision.

### Optic Surfaces for the Optic Zone Regions

To provide the desired optical powers for the optic zone regions, either conic refractive profiles can be used, or diffractive profiles can be used.

In the case of simple conic refractive profiles, each optic zone provides its optical power via a conic curve such that the apical radius of curvature provides the desired optical power and the conicity (K) value is set to reduce spherical aberrations for the region. Optimization to find the apical radius and the conicity can be done numerically using commercially available optical design programs such as Zemax or using closed form analytical equations. Both of these methods are known to those skilled in the art. Additionally, the conicity value can be adjusted to further enhance the depth of field performance of the IOL. Conicity values in the range of −7.5 to −9.5 and typically, −8.717 provide such an enhancement for a equal biconvex conic optic zone.

When simple conic refractive profiles are used and the central region 9 of the optical zone provides distance vision and the peripheral region 10 provides near vision, the transition between the regions is negligibly small. This is the preferred arrangement as transition regions generally cause stray light that would otherwise be properly focused by one of the two optical power regions.

When simple conic refractive profiles are used and the central region 9 of the optical zone provides near vision and the peripheral region 10 provides distance vision, the transition between the regions is required to smoothly join the regions. This transition profile is generally implemented by either a Bezier curve or a circular fillet, both of which are known to those skilled in the art.

### Peripheral Add Zone

In another embodiment the add zone can be placed inside the virtual aperture region. In still another embodiment the add zone can be placed on the posterior side inside the large transition region. The peripheral add zone could be present along with the add zone in the central optic.

### Cylinder Power to Correct Astigmatism

To correct for astigmatism, a cylinder component can be added to one or both surfaces of the IOL optic zone. The cylinder power for this purpose is in the range of 0.5 to 6.0 diopters in steps of either 0.25 or 0.5 D.

To use the concepts described above to define a surface of the IOL, the following is done. First, the central optic of the IOL is specified as explained above. The diameter of the optic zone is around 1.5 mm and such as between (1.4 and 1.6 mm). Optical powers for this optic zone vary from −10 to 40 D in steps or 0.25 or 0.5 D. Cylinder powers for toric IOLs vary from 0.5 to 6.0 D in steps of 0.25 to 0.5 D.

The virtual aperture is then generated using the concepts described in previous disclosures. The width of the virtual aperture region is about 2.0 mm.

The width of the front surface transition regions is each set to around 0.15 mm. The width of the back-surface transition region is set to around 2.3 mm.

The design of the haptic is considered a separate issue and is routine for those skilled in the art.

Once the front and back surfaces have been specified, individual profile samples are taken from the center of the IOL to the periphery to specify the points for the lathe cutting file.

### Subsurface Modifications of IOL

In an embodiment, at least one region of the IOL, such as the virtual aperture 48 of the IOL, includes at least one subsurface modification comprising a modification to at least a portion of the internal structure of the IOL. The IOL can include such a subsurface modification as well as an optional external surface feature (such as a shape change or contour on the external surface) on an anterior and/or posterior external surface of the IOL. The subsurface modification is configured to achieve a desired optical effect on light that passes therethrough or otherwise interacts with the subsurface modification, such as to diffuse light, homogenize light, or redirect light for example. The subsurface modification of the IOL provides an alternate, efficient, and repeatable mechanism for at least one region of the IOL to diffuse and/or homogenize light passing therethrough. A degree or level of diffusion and/or homogenization can be tailored to specific requirements by varying the size of laser damage spots or a modified refractive index loci as described below. The spacing or density of the placement of the damage spots or loci can be varied as can a quantity of layers of such damage spots or loci to achieve a desired level of light diffusion. The configuration of the damage spots or loci can also be used to achieve directional control of light such as to steer light in a desired direction. This enables fine tuning and customization of the optical properties of the IOL or of a light diffuser device. The intraocular lens can be part of a system including a laser emitting device configured to emit a laser on a material pursuant to formation of an IOL.

In any embodiment, the loci can intersect the anterior surface or posterior surface (such as by being positioned tangential to the respective surface.) Or the loci can be positioned at any depth relative to the anterior surface or posterior surface including the surface itself.

The device can achieve diffraction of light passing therethrough in a variety of manners via a diffractive feature contained within or coupled to the device such as the IOL. The diffractive feature is sized to be sufficiently small to create a diffractive effect on light rays interacting with the diffractive feature to widely disburse the light on the retina (or other object.) The diffractive feature can be, for example, a subsurface modification, a prism (or portion thereof such as an edge, point or apex of a prism), a step-shape, a hole or aperture in the IOL, and/or a mask positioned on the IOL. The device can also be configured to achieve diffraction of light via a diffractive feature positioned on or over the device.

In an embodiment, the subsurface modification(s) are not positioned in the virtual aperture but are rather part of an optical correction zone of the IOL, which may or may not be in the virtual aperture 48 region of the IOL. In another embodiment, the subsurface modifications form a light diffusion region of an IOL or of a light transmitting body or structure that is not an IOL. For example, the features described herein can be used in a light diffuser device that is not an IOL.

In a first example embodiment of a subsurface modification, a laser is configured to interact with an internal region (i.e., a subsurface region or location) of the IOL to achieve the subsurface modification, such as a modification to the structure of the IOL at the subsurface location. The same laser or different laser can also interact with a surface region of the IOL such that a first laser interacts with a surface region while a second or different laser interacts with a subsurface region. The subsurface region is positioned between at least an anterior surface and a posterior surface of the IOL. In an example, a laser is focused below the surface of the IOL such as to heat the material of the IOL and form a damage region or damage spot located within the material of the IOL at a subsurface location.

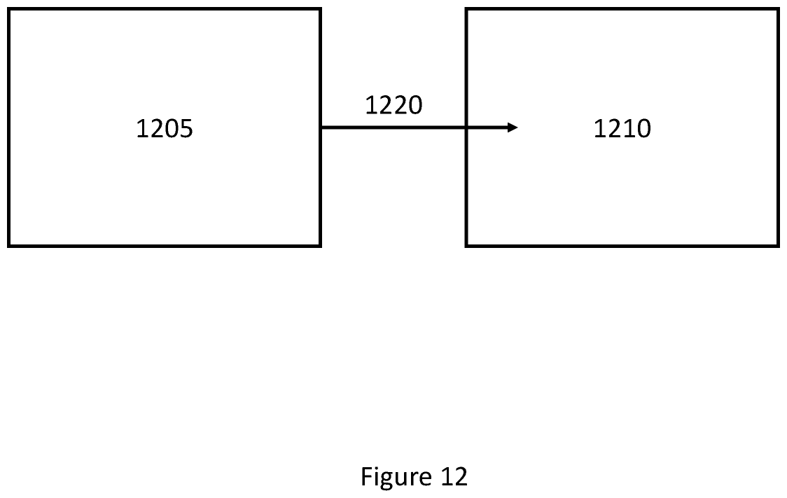

FIG. 12 shows a schematic representation of a laser system 1205 that is configured to interact with an IOL 1210 (or with a piece or body of material that is subsequently formed into, placed onto, or otherwise incorporated into the IOL 1210 or that forms a device that is not an IOL such as a light diffuser device.) The laser system 1205 is configured to emit a laser 1220 that interacts with the IOL, such as laser 1220 that focuses or otherwise emits a predetermined amount of energy at a subsurface location of the IOL 1210.

The laser system 1205 is configured to emit the laser 1220 such that the laser 1220 is focused below the surface of the IOL material (such as a glass or polymer material in a non-limiting example) or that is configured to emit a predetermined level of energy at a subsurface location. In an embodiment, the laser is pulsed at a high rate. The laser 1220 creates one or more microscopic damage points inside (i.e. below an external surface of or between an anterior surface and posterior surface of) the IOL material. In an example embodiment, the pulsed laser causes rapid material heating and expansion in a vicinity of the focused laser spot, which create stresses and small-scale fracturing and gas expansion of the material to thereby form a damage spot. The resultant fracture or damage spot can have extremely small dimension (such as on the order of 10s of microns).

The laser can be moved rapidly and accurately in a lateral X/Y direction while focused at a particular depth (Z-direction) in the material relative to an anterior or posterior external surface. A pattern or array of such damage spots can be formed at the depth. In addition, two or more layers of such damage spots can be formed. The depth of the laser focus spot(s) is accurately and rapidly controlled such as to a depth resolution on a micron scale.

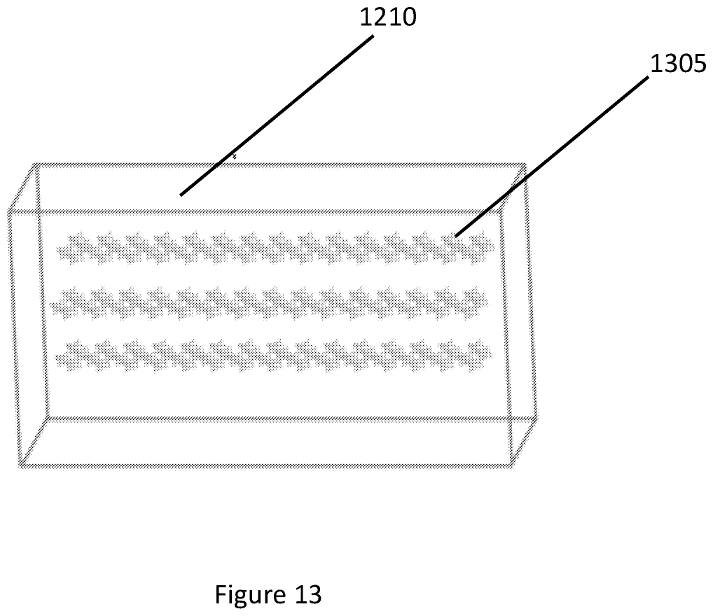

The laser thus forms a two- or three-dimensional array of damage spots that can be arranged in any of a wide variety of patterns. A two-dimensional array includes two or more damage spots positioned in a common plane. A three-dimensional array includes two or more two-dimensional arrays. FIG. 13 shows a schematic representation of a portion of the IOL 1210. It should be appreciated that the portion of the IOL 1210 in FIG. 13 is represented as a prism shape for ease of illustration although the shape can vary and is not limited to a prism shape. A two- or three-dimensional array of damage spots 1305 is positioned entirely below an external surface of the IOL 1210. The array includes one or more damage spots. In the illustrated example, the damage spots form a rectangular-shaped array of equidistant damage spots although the shape and spatial arrangement of the array and the damage spots within the array can vary.

In an example fabrication process for an IOL, the following steps can be performed. First, an IOL is formed such as on a lathe from a plastic (or other material) blank using any well-known process for forming an IOL. The IOL can be machined of any of a variety of materials with an optical zone in the central portion that is configured to enable extended depth of field or monocular focusing. In an embodiment, the IOL is configured having the features described herein with reference to FIGS. 6A-7. Next, the virtual aperture can be formed having flat posterior and anterior surfaces (i.e., the outer surface is not machined or otherwise modified) or the anterior or posterior surfaces can be machined to include desired surface features, such as grooves, ridges, waves, ripples, prisms, or any other surface feature. Next, one or more haptics are machined into the substrate blank according to specifications to allow surgical implantation and proper placement in the eye.

The laser system 1205 is then employed to create a 2 dimensional or 3 dimensional pattern of damage spots within the virtual aperture of the IOL as described above. That is, the damage spots can be aligned within a common plane. In another embodiment, the IOL includes a series of planes arranged to form a three-dimensional array of planes wherein each plane includes one or more damage spots.

An alignment process and/or system can be employed to properly align the IOL so that the laser damage is aimed correctly and precisely. The two- or three-dimensional array of damage spots is configured to enable a prescribed or desired amount of light transmission and diffusion therethrough. For example, the pattern can be a 5-10 layer pattern of 50 micron spots arranged in a rectangular grid or annular grid with 50 micron spacing between damage spots. The pattern can include an offset between layers such that the gaps are filled in when viewed axially. Since a uniform distribution of damage spots can lead to visual artifacts when implanted in an eye, an exemplary spots pattern employs a pseudo-random placement strategy.

In a second example embodiment of a subsurface modification, a femtosecond pulsed laser (FSPL) is configured to interact with the IOL (such as by being focused at a subsurface location of the IOL) to modify a refractive index of one or more subsurface locations of the IOL. The femtosecond pulsed laser forms modified loci in the subsurface locations wherein the modified loci have a different refractive index than the refractive index of the material before modification. Different patterns of modified loci can provide selected dioptic power, toric adjustment, and/or aspheric adjustment provided. The refractive index of the modified loci can also be different from a refractive index at a subsurface location that surrounds the modified loci. The different refractive index may be caused by nonlinear absorption of photons resulting from exposure to focused laser light via the femtosecond pulsed laser.

With reference again to FIG. 12, the laser system 1205 can be configured to emit a femtosecond pulsed laser 1220. The femtosecond laser is focused to a point below the surface of the IOL 1210 and is pulsed in a very specific time and intensity profile. The laser can be controlled in the XY plane, such as by using a Galvo positioning controller, which enables very high speed and high accuracy placement of the beam in the XY plane. Additionally, the system can be coupled to or otherwise use an acoustically controlled focusing mechanism enabling very high frequency and very high accuracy focusing control. This enables positioning of the femtosecond laser focus spot at any depth in the substrate and at any XY coordinate with extreme speed and precision.

The femtosecond laser pulses affect the internal region of the IOL so as to change the refractive index of a specific subsurface region of the IOL and form the loci. The process can be employed with a wide variety of IOL materials including, for example, glass, hydrophobic and hydrophilic acrylics. The mechanism resulting in the change in refractive index is different in each substrate but in all of the substrates mentioned the laser effected area will have a lower refractive index than the surrounding material. The decrease in refractive index may be dependent on various factors including the specifics of the substrate, the intensity and duration of the laser exposure and the thickness of the material. In general, a change in refractive index of about 0.06 is consistently achievable. For example, if the original hydrophilic acrylic substrate has a nominal refractive index of 1.459 in its fully hydrated configuration, then after laser exposure, the treated areas could have a refractive index as low as 1.399.

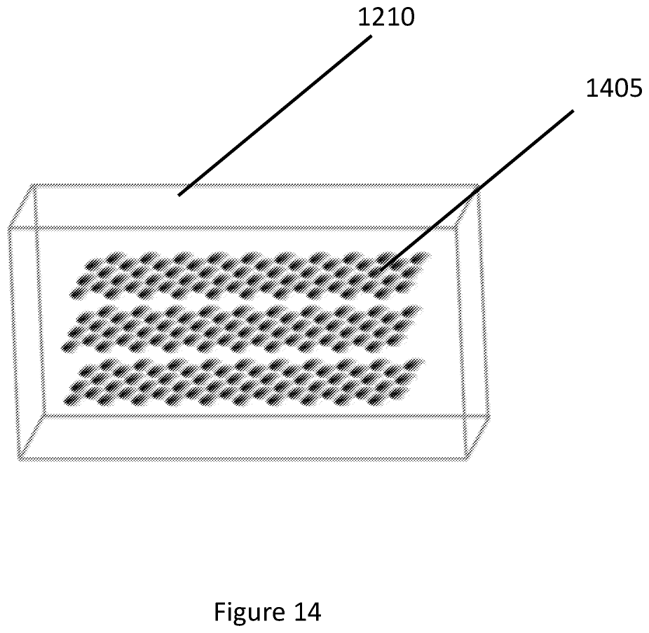

FIG. 14 shows a schematic representation of a portion of the IOL 1210. An array of loci 1405 (each having a modified refractive index) is positioned entirely below an external surface of the IOL 1210. The array includes one or more loci. In the illustrated example, the loci form a rectangular-shaped array of equidistant loci although the shape and spatial arrangement of the array and the loci within the array can vary.

In a sample manufacturing process, the IOL is formed using a lathe to form an IOL with a virtual aperture as described herein. With reference to FIG. 12, the IOL is aligned with a femtosecond laser apparatus 1205, which emits femtosecond laser 1210 focused at a subsurface location to create the a desired subsurface pattern of modified refractive index zones to achieve desired diffusion, transmission, and beam steering.

In any embodiment of subsurface modification, multiple layers of the damage spots or loci within an array permits spreading and homogenization of a narrow beam if light that passes into the IOL.

To the extent the IOL or a portion of the IOL diffuses light, such diffusion can be achieved by various features or techniques associated with the IOL or associated with manufacture of the IOL. Such techniques can include one or more surface modifications of the IOL such a modifying a surface of the IOL using an annular lathe. An annular or non-annular surface modification including a micro-lens can be used. Such modifications can be randomly or pseudo-randomly positioned on the IOL. In another embodiment, a surface of the IOL is abraded (such as via sandblasting, polishing with a specific grit) such as to randomly roughen the surface or to achieve a desired surface roughness. The surface can also be chemically etched or roughened using a laser such as to selectively burn away surface material. Any combination of the aforementioned techniques can also be used.

In another implementation, diffusion is achieved by modifying an internal aspect of the IOL while leaving an external surface unmodified (or in combination with modification of an external surface of the IOL.) The IOL can incorporate a holographic diffuser wherein a holographic diffuser interference pattern is either sandwiched inside the IOL or formed directly during manufacture of the IOL such as during polymer curing. The IOL may also or alternatively employ a milky material such as a combination of two or more diffusive materials that are homogeneously translucent but not transparent. This can allow for more light attenuation and scattering of light. Subsurface laser engraving can also be employed to achieve diffusion.

The IOL can be manufactured pursuant to various processes and devices including a lathe, injection molding, a sandwich construction, an ablative laser, a mask laser, and use of a cured polymer. Subsurface laser marking, embossing, a glass embossing plate, silicon molding and surface casting can also be used. Chemical etching can also be used to modify a surface of the IOL.

While this specification contains many specifics, these should not be construed as limitations on the scope of an invention that is claimed or of what may be claimed, but rather as descriptions of features specific to particular embodiments. Certain features that are described in this specification in the context of separate embodiments can also be implemented in combination in a single embodiment. Conversely, various features that are described in the context of a single embodiment can also be implemented in multiple embodiments separately or in any suitable sub-combination. Moreover, although features may be described above as acting in certain combinations and even initially claimed as such, one or more features from a claimed combination can in some cases be excised from the combination, and the claimed combination may be directed to a sub-combination or a variation of a sub-combination. Similarly, while operations are depicted in the drawings in a particular order, this should not be understood as requiring that such operations be performed in the particular order shown or in sequential order, or that all illustrated operations be performed, to achieve desirable results. Only a few examples and implementations are disclosed. Variations, modifications and enhancements to the described examples and implementations and other implementations may be made based on what is disclosed.

1. An intraocular lens configured to provide an extended depth-of-field, said intraocular lens comprising:
an optical zone comprising at least one anterior optical surface and at least one posterior optical surface;
a first periphery region peripherally positioned relative to the optical zone, the first periphery region comprising a virtual aperture, the virtual aperture comprising an anterior virtual aperture surface and a posterior virtual aperture surface, wherein the virtual aperture comprises a first subsurface region having a first refractive index, and wherein the virtual aperture further comprises a plurality of modified, subsurface loci, wherein the modified, subsurface loci have a second refractive index, which is different from the first refractive index and caused by nonlinear absorption of photons resulting from exposure to focused laser light; and
a second periphery region peripherally positioned relative to the first periphery region, the second periphery region comprising a haptic for positioning the intraocular lens within an eye, wherein the haptic comprises an outermost region of the intraocular lens;
wherein a first plurality of light rays incident on the anterior optical surface pass through the optical zone to form an image on a retina when the intraocular lens is implanted in an eye; and
wherein a second plurality of light rays incident on the anterior virtual aperture surface are dispersed widely downstream from the intraocular lens towards and across the retina, such that the image comprises the extended depth-of-field and further wherein said virtual aperture reduces monochromatic and chromatic aberrations in the image.

2. The intraocular lens of claim 1, wherein the anterior or posterior surface of the virtual aperture comprises a hexagonal micro-structure having an outer boundary defined by a hexagon.

3. The intraocular lens of claim 1, wherein the anterior or posterior surface of the virtual aperture comprises a hexagonal micro-structure having a micro-lens.

4. The intraocular lens of claim 3, wherein at least one micro-lens comprises a convex sphere.

5. The intraocular lens of claim 3, wherein at least one micro-lens comprises a concave sphere.

6. The intraocular lens of claim 3, wherein at least one micro-lens comprises a conicoid.

7. The intraocular lens of claim 1, wherein the first periphery region is connected to the central optical zone by a first transition region.

8. The intraocular lens of claim 6, wherein the second periphery region is connected to the first periphery region by a second transition region.

9. The intraocular lens of claim 1, wherein the optical zone includes at least two discrete regions including a first discrete region and a second discrete region.

10. The intraocular lens of claim 8, wherein the first discrete region is a central region and the second discrete region is a peripheral region positioned peripherally around the central region.

11. The intraocular lens of claim 8, wherein the first discrete region comprises a first distance power and the second discrete region comprises a second distance power.

12. The intraocular lens of claim 1, wherein the first periphery region includes a plurality of holes of predetermined size, wherein the plurality of holes is collectively configured to permit the passage of light therethrough to create a diffractive effect.

13. The intraocular lens of claim 1, wherein the optical is 1.5 to 3.3 mm in size.

14. The intraocular lens of claim 1, wherein at least one subsurface loci is configured to create a diffractive effect on light passing therethrough.

15. The intraocular lens of claim 1, wherein the plurality of modified, subsurface loci is arranged in a two dimensional array of loci.

16. The intraocular lens of claim 1, wherein the plurality of modified, subsurface loci is arranged in a three dimensional array of loci.

17. The intraocular lens of claim 16, wherein the subsurface loci form a rectangular shaped array of equidistant loci.

18. The intraocular lens of claim 1, wherein the second refractive index is different from the first refractive index by 0.06.
